(19) United States
(12) Patent Application Publication
(10) Pub. No.: US 2025/0086314 A1
(43) Pub. Date: Mar. 13, 2025

(54) STREAM DE-IDENTIFICATION THAT SUPPORTS MEDICAL DATA STANDARDS

(71) Applicant: Signify Health, LLC, Dallas, TX (US)

(72) Inventors: Sean Thomas Boylan, Galway (IE); Philip Johnson, Secaucus, NJ (US); Milosz Dominik Milewski, Galway (IE); Nino Musanovic, Galway (IE); Conor Killilea, Oranmore (IE)

(21) Appl. No.: 18/463,877

(22) Filed: Sep. 8, 2023

(51) Int. Cl. G06F 21/62; G16H 10/60

(52) U.S. Cl. CPC G06F 21/6254; G16H 10/60

(57) ABSTRACT

A system can include one or more memory devices storing instructions thereon, that, when executed by one or more processors, cause the one or more processors to receive a message of a message or event stream, the message including a medical data or other data. The instructions cause the one or more processors to parse the message into a plurality of components representing personal identifying information of the medical data. The instructions cause the one or more processors to transform the plurality of components to de-identify the personal identifying information. The instructions cause the one or more processors to write a second message based on the plurality of transformed components.

### INTRODUCTION

Medical data can include personal identifying information (PII) of patients. Some systems may not have authorization to store or access the PII.

### SUMMARY

At least one example is directed to a system. The system can include one or more memory devices storing instructions thereon, that, when executed by one or more processors, cause the one or more processors to receive a message or event of a message stream, the message including medical and/or other data types. The instructions can cause the one or more processors to parse the message into components representing personal identifying information of the medical data. The instructions can cause the one or more processors to transform the components to de-identify the personal identifying information. The instructions can cause the one or more processors to write a second message based on the transformed components.

The instructions can cause the one or more processors to stream the second message, and second messages including transformed components, to a second system.

The instructions can cause the one or more processors to transform a first component of the components. The instructions can cause the one more processors to write the transformed first component to the second message. The instructions can cause the one or more processors to transform a second component of the components as the transformed first component is written to the second message. The instructions can cause the one or more processors to write the transformed second component to the second message.

The instructions can cause the one or more processors to write the second message based on the transformed components as the components are transformed.

The instructions can cause the one or more processors to identify a component of the components that is excluded from transformation. The instructions can cause the one or more processors to write the component to the message in the clear.

The instructions can cause the one or more processors to add salt values to the components. The instructions can cause the one or more processors to transform the components with the salt values.

The instructions can cause the one or more processors to hash a component of the components. The instructions can cause the one or more processors to determine that a number of characters of the hashed component is greater than a threshold. The instructions can cause the one or more processors to discard at least one character of the characters of the hashed component to cause the number of characters of the hashed component to equal the threshold.

The instructions can cause the one or more processors to identify that a component of the components represents a type of personal identifying information of types of personal identifying information. The instructions can cause the one or more processors to select a rule for the type from rules, the rules linked with the types of personal identifying information. The instructions can cause the one or more processors to format a transformed component of the transformed components based on the rule by adding at least one additional character to the transformed component or replacing at least one character of the transformed component with a predefined type of character.

The instructions can cause the one or more processors to add the second message to an inbox of a first environment, the inbox including second messages. The instructions can cause the one or more processors to receive a trigger. The instructions can cause the one or more processors to move the second messages from the inbox to an outbox of a second environment.

The instructions can cause the one or more processors to detect that there is no formatting rule for a personal identifying information type a component of the components. The instructions can cause the one or more processors to detect a data type of the component. The instructions can cause the one or more processors to format a transformed component of the transformed components based on a default rule of default rules, the default rules linked to data types.

At least one example is directed to a method. The method can include receiving, by one or more processing circuits, a message of a message stream, the message including a medical data. The method can include parsing, by the one or more processing circuits, the message into components representing personal identifying information of the medical data. The method can include transforming, by the one or more processing circuits, the components to de-identify the personal identifying information. The method can include writing, by the one or more processing circuits, a second message based on the transformed components.

The method can include transforming, by the one or more processing circuits, a first component of the components. The method can include writing, by the one or more processing circuits, the transformed first component to the second message. The method can include transforming, by the one or more processing circuits, a second component of the components as the transformed first component is written to the second message. The method can include writing, by the one or more processing circuits, the transformed second component to the second message.

The method can include identifying, by the one or more processing circuits, a component of the components that is excluded from transformation. The method can include writing, by the one or more processing circuits, the component to the message in the clear.

The method can include adding, by the one or more processing circuits, salt values to the components. The method can include transforming, by the one or more processing circuits, the components with the salt values.

The method can include hashing, by the one or more processing circuits, a component of the components. The method can include determining, by the one or more processing circuits, that a number of characters of the hashed component is greater than a threshold. The method can include discarding, by the one or more processing circuits, at least one character of the characters of the hashed component to cause the number of characters of the hashed component to equal the threshold.

The method can include identifying, by the one or more processing circuits, that a component of the components represents a type of personal identifying information of types of personal identifying information. The method can include selecting, by the one or more processing circuits, a rule for the type from rules, the rules linked with the types of personal identifying information. The method can include formatting, by the one or more processing circuits, a transformed component of the transformed components based on the rule by adding at least one additional character to the transformed component or replacing at least one character of the transformed component with a predefined type of character.

The method can include adding, by the one or more processing circuits, the second message to an inbox of a first environment, the inbox including second messages. The method can include receiving, by the one or more processing circuits, a trigger. The method can include moving, by the one or more processing circuits, the second messages from the inbox to an outbox of a second environment.

The method can include detecting, by the one or more processing circuits, that there is no formatting rule for a personal identifying information type of a component of the components. The method can include detecting, by the one or more processing circuits, a data type of the component. The method can include formatting, by the one or more processing circuits, a transformed component of the transformed components based on a default rule of default rules, the default rules linked to data types.

One or more storage medium storing instructions thereon, that, when executed by one or more processors, cause the one or more processors to receive a message of a message stream, the message including a medical data. The instructions can cause the one or more processors to parse the message into components representing personal identifying information of the medical data. The instructions can cause the one or more processors to transform the components to de-identify the personal identifying information. The instructions can cause the one or more processors to write a second message based on the transformed components.

The instructions can cause the one or more processors to transform a first component of the components. The instructions can cause the one or more processors to write the transformed first component to the second message. The instructions can cause the one or more processors to transform a second component of the components as the transformed first component is written to the second message. The instructions can cause the one or more processors to write the transformed second component to the second message.

All examples and features mentioned above can be combined in any technically possible way.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is an example system that de-identifies a medical message stream.

FIG. 2 is an example of de-identification of a medical message stream communicated between environments.

FIG. 3 is an example of de-identification of a message.

FIG. 4 is an example method of de-identification of a medical message stream.

FIG. 5 is another example method of de-identification of a medical message stream.

FIG. 6 is an example computing architecture of a data processing system.

### DETAILED DESCRIPTION

Following below are more detailed descriptions of various concepts related to, and implementations of, methods, apparatuses, and systems of medical stream de-identification. The various concepts introduced above and discussed in greater detail below may be implemented in any of numerous ways.

A system can receive medical data from one or more data sources, such as hospital data from a hospital system, ambulance data from an ambulance system, records data from a record database, a nursing facility, health card record system, etc. The system can receive the medical data as messages generated responsive to data being created, or an event occurring. The system can receive data from many different data sources simultaneously. Medical events can include PII of patients, users, clinicians, or families. One system, platform, environment, or device that communicates the medical events may need to de-identify the PII. For example, a first system, platform, environment, or device that transmits the medical events to a second system, platform, environment, or device may need to de-identify the PII before transmitting the medical events to the second system. The second system may not be authorized to view or store the PII, e.g., in order to comply with one or more guidelines.

The system can store a database of medical events. The system can de-identify the medical events stored in the database, and then transmit the de-identified medical events to the second system. However, this batch processing approach can include multiple technical problems. First, the batch processing approach may not be usable when a stream of messages is received by the first system and needs to be communicated to the second system in real-time, or near real-time (e.g., with minutes of delay, with seconds of delay or milliseconds of delay). Furthermore, the system can receive thousands of messages, hundreds of thousands of messages, or millions of messages. The batch processing approach can introduce significant delay in de-identifying and communicating the stream of messages across the systems when the volume of messages received is significantly large.

Second, the batch processing approach can be static and have flexibility and, in some instances, may have privacy based issues. The batch processing approach can be designed to work with a specific database format. The batch processing approach can be designed to de-identify PII of a particular set of rows or columns of the database. However, if the structure of the database changes, e.g., a new column or row is added to the database, and PII is stored in the new column or row, the solution may not de-identify the PII of the new row or column, and therefore, PII could be exposed if the solution does not automatically adapt to changes in the structure of the database.

To solve for these, and other technical issues, this technical solution can de-identify streamed messages. The system can de-identify messages of a stream in real-time, or in near real-time (e.g., in minutes, seconds, or milliseconds) and handle a large volume of messages (e.g., thousands of messages, hundreds of thousands of messages, millions of messages) in a variety of different message formats. As messages are received, the system can parse each message to break the message into multiple components, at least one of the components including PII. The system can transform each component (e.g., with hashing or encryption) and write the transformed components to a second message. The system can transform each component with a non-reversable transformation, such as hashing. In some implementations, the system can transform each component with a reversible transformation, such as encryption. The system can stream the second message to another system or platform. The transformed components can be transformed in a deterministic and non-reversable manner. For example, each transformed component can be hashed, such that the resulting hash of each component can provide an index for the PII, but cannot be used to identify the PII used to generate the hashed component.

Unless a component is excluded from the transformation, the system can apply the transformation across all of the components of the message, such that no PII that needs to be kept hidden is left exposed or in the clear. The system can store a configuration file that identifies which components should be excluded from transformation. Unless a configuration file indicates that a component should not be de-identified, the system de-identifies all components of the message. By de-identifying all the components of the message, new or different types of components or message formats that the system receives will still be properly de-identified, and no PII will unintentionally be left exposed or in the clear. In this regard, the drawbacks of identifying which fields to de-identify is solved by always transforming all components of a message, unless the configuration file explicitly indicates otherwise.

The system can write the transformed component to the second message. The messages can be written in real-time as the components are transformed, and therefore, the transformation and message writing can occur together, resulting in increased speeds for de-identifying and streaming the messages.

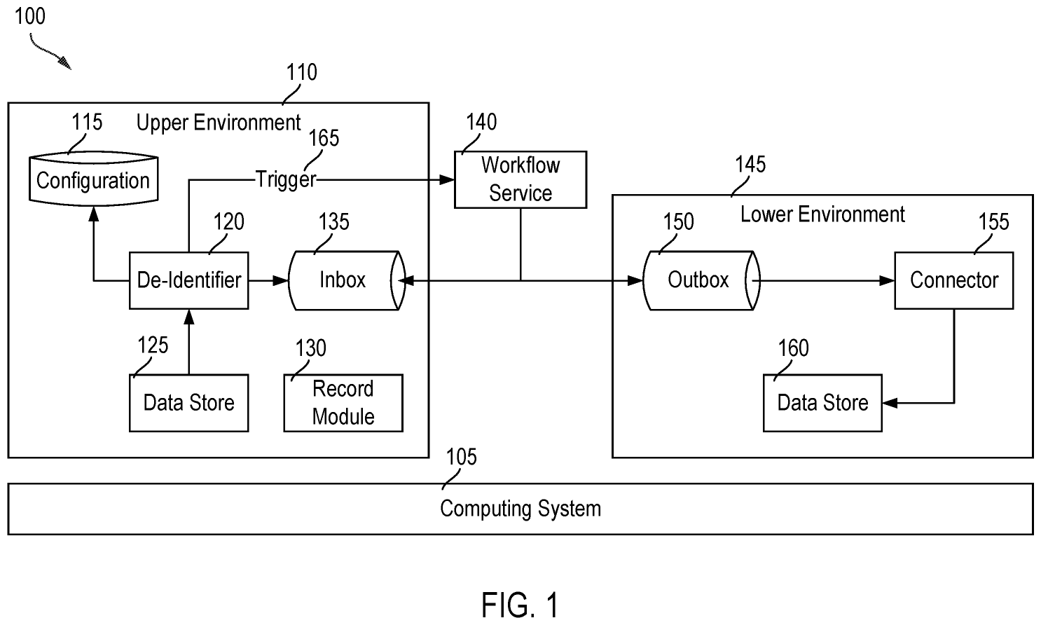

Referring now to FIG. 1, among others, an example system 100 that de-identifies a medical message or event stream is shown. The system 100 can be a network of devices or systems, collection of devices or systems, or a group of devices or systems. The system 100 can include at least one computing system 105. The computing system 105 can be one or multiple computers, computer systems, servers, server systems, cloud platforms, etc. The computing system 105 can include one or multiple processors, memory devices, processing circuits, processing circuitry, hard drives, and/or data storage. The computing system 105 can be distributed across multiple physical locations, e.g., across different computing systems located in different geographic locations. The computing system 105 can be located at a single physical location.

The computing system 105 can implement at least one upper environment 110 and at least one lower environment 145. The upper environment 110 and the lower environment 145 can be a first and second environment. The upper environment 110 can be authorized to receive, store, or process messages including PII. However, the lower environment 145 may not have, or may lack, the authorization to receive, store, or process messages including PII. Therefore, the upper environment 110 can de-identify messages before the messages are transmitted or moved to the lower environment 145. The environments 110 and 145 can be computing environments, collections of software, collections of computing systems, applications, or other software constructs. The upper environment 110 can be a production environment, a user acceptance environment, or a quality assurance environment. The lower environment 145 can be a user acceptance environment, a quality assurance environment, or a development environment. The upper environment 110 and the lower environment 145 can be first and second systems, first and second devices, or first and second computing environments.

The upper environment 110 can include at least data store 125. The data store 125 can be a topic or raw topic. The data store 125 can be a virtual group of messages or events or a virtual log of messages or events. The data store 125 can store or organize the messages or events. The data store 125 can allow producers to write messages to the data store 125, and consumers to read messages from the data store 125. The data store 125 can be a Kafka topic, or may be another type of data store. The data store 125 can include a name or identifier that a system, device, or apparatus can send or write messages to. A consuming or subscribed device, component, or system can receive or read the messages from the topic 125, e.g., the de-identifier 120 can be a consumer of the data store 125. The de-identifier 120 can be registered or subscribed to the data store 125 to receive messages of at least one medical message stream sent to the data store 125. The data store 125 can be a topic for a stream of medical messages or medical events. Streamed messages can be of a predefined length or size, and therefore may be suitable for real-time processing. Furthermore, the streamed messages can adhere to a specific structure or standard, and therefore can be suitable for parsing, transforming, and de-identifying a stream of messages.

The messages can be received from a variety of data sources that produce medical events or messages that include PII (e.g., names, addresses, phone numbers, email addresses, medical test data). The messages can include data of a medical event. The data sources can be or include hospitals, ambulance systems, nursing facilities, health care provider record systems, electronic health record (EHR) systems, or retirement homes. The data can be messages, events, or pieces of data that include Health Level 7 (HL7) data (e.g., admissions data), fast healthcare interoperability resource (FHIR) data, discharge or transfer events used to communicate patient acute clinical events between systems (e.g., between healthcare facilities). The messages can be or include JavaScript Object Notation (JSON) data, extensible markup language (XML) data, binary data, or any other kind of data in any kind of data format. The messages can be pipe delimited messages. The messages can by any type of HL7 v2, HL7 v3, HL7 v4, or generic XML or JSON format.

The upper environment 110 can include at least one de-identifier 120. The de-identifier 120 can be a registered consumer of the data store 125, and can receive messages of a message stream written to the data store 125. The de-identifier 120 can de-identify the messages in real-time, or as the messages are received, and provide the messages to at least one inbox 135. The upper environment 110 can include at least one configuration 115. The configuration 115 can include at least one file, data structure, database, template, or other software storage component. The configuration 115 can include a processing configuration indicating different components in different types of messages. The configuration 115 can indicate formatting rules for formatting different types of transformed components. The configuration 115 can be reviewed and validated by an entity or user before the configuration 115 is deployed for use in de-identifying messages.

The de-identifier 120 can parse messages received from the data store 125. The de-identifier 120 can parse the message into one or multiple components. Each component can represent PII of a medical event or metadata describing the message. A component can represent PII included in the message based on the medical event generated at the data source. The de-identifier 120 can identify the message component type and select a parsing rule from the configuration 115 that identifies the components in the message. For example, for a JSON message, the configuration 115 can indicate components of the JSON message. For an XML message, the configuration 115 can indicate components of the XML message. The configuration 115 can be specific to a source that produced a message. For example, messages from a hospital may have one or more particular components that the de-identifier 120 can parse. Messages from an ambulance system can have one or more particular components that the de-identifier 120 can parse.

The de-identifier 120 can transform each or a set of components parsed from the message. The de-identifier 120 can receive a rule or set of rules from the configuration 115 that identifies one or more different components that should not be transformed, and instead output in the clear or without obfuscation. For example, based on the message type, the de-identifier 120 can identify whether components parsed from the message are excluded from transformation. If the component is excluded from transformation, the de-identifier 120 can write the component to an output message in its original form, e.g., in the clear, without obfuscation, without transformation. The remaining components that are not excluded from transformation, can be transformed to de-identify the PII represented in the components. The transformation can hide or obfuscate the PII in the components (e.g., with hashing or encryption).

The de-identifier 120 can transform each component by salting and/or hashing the components. The de-identifier 120 can add at least one salt value to each component of the message. The de-identifier 120 can transform at least one component with the salt values. For example, the de-identifier 120 can apply a salt value to each component, e.g., concatenate each component with a salt value. For example, the salt value can be concatenated with the component at the beginning of the component (e.g., at the front of the component) or at the end of the component. The de-identifier 120 can hash each component with its salt value. The salt value can be a pseudo-randomly generated value or a predefined value. The de-identifier 120 can apply a SHA512, SHA-256, MD5, RIPEMD-160, Whirlpool, or any other type of hashing algorithm. Hashing functions can be fast and non-reversible, guaranteeing proper de-identification. Applying a hashing function can guarantee the integrity of message data within and across messages in the stream. A given message component's value can always hash to the same value, so long as the same salt and hashing algorithm is applied. This means that the de-identified data will continue to make sense within and across messages, and therefore maintain the meaningful and usefulness of the de-identified data.

The de-identifier 120 can write a second message based on the transformed components of the message. The de-identifier 120 can write the second message as the components are transformed. For example, the de-identifier 120 can write each transformed component as soon as the component is transformed, instead of waiting for all of the components to be transformed and then writing the message. For example, the de-identifier 120 can transform a first component, and then write the transformed first component to the second message. The first component can be transformed and written to the second message even before a second component is transformed or written or as the second component is transformed or written. The second component can be transformed, e.g., transformed as the first component is written to the second message. Then, the second component can be written to the second message. In this regard, the message can be transformed and written quickly, e.g., less than a second, less than 500 milliseconds, less than 100 milliseconds, less than 10 milliseconds, less than 1 millisecond. In this regard, the de-identifier 120 can de-identify the messages received via the data store 125 in real-time.

The de-identifier can write, provide, add, or transmit the de-identified messages to at least one inbox 135. The upper environment 110 and the lower environment 145 can implement an inbox-outbox pattern. The inbox 135 can include a unique identifier, and can include storage for storing de-identified messages. As the de-identifier 120 writes de-identified messages, the de-identifier 120 can write the message to the inbox 135. The inbox 135 can be a blob storage inbox account for the upper environment. The lower environment 145 can include an outbox 150. The outbox 150 can be a blob storage account for the lower environment 145. The outbox 150 can store one or multiple messages received from the inbox 135. The inbox 135 and the outbox 150 can both be blob storage, or any other kind of unstructured storage.

A workflow service 140 can move messages between the upper environment 110 and the lower environment 145 by moving the messages from the inbox 135 to the outbox 150. The workflow service 140 can receive a trigger 165, which can be a command to move the messages from the inbox 135 to the outbox 150. The trigger 165 can be generated by the upper environment 110, e.g., the de-identifier 120. The workflow service 140 can cause the messages to be moved from the inbox 135 to the outbox 150 responsive to the trigger 165. The de-identifier 120 can generate the trigger 165 and provide the trigger 165 to the workflow service 140 to move the messages between the inbox 135 and the outbox 150 responsive to a condition. The condition can be a period of time elapsing, e.g., more than ten seconds, five to ten seconds, one to three seconds, 500-900 milliseconds, 100-500 milliseconds, 10-100 milliseconds, less than 10 milliseconds, less than 1 millisecond, etc. The de-identifier 120 can monitor the inbox 135 and generate the trigger 165 responsive to a number of messages in the inbox 135 reaching a level or exceeding the level. The level or threshold can be 50-100 messages, 10-150 messages, less than 10 messages, more than 150 messages.

The lower environment 145 can include at least one connector 155 and at least one data store 160. The connector 155 can connect the outbox 150 with the data store 160. The connector 155 can read the outbox 150 for messages, and write the messages to the data store 160. Consuming applications, systems, or devices of the lower environment 145 can receive the messages based on the connector 155 writing the messages of the outbox 150 to the data store 160. For example, the connector 155 can write the messages of the outbox 150 to the data store 160 as soon as the messages are moved into the outbox 150 or immediately after the messages are moved into the outbox 150, e.g., within 1 second, within 500 milliseconds, within 10 milliseconds, within 1 millisecond. The data store 160 can be or provide a message stream of the messages in the lower environment 145.

Overall, the messages can be streamed from the data store 125 of the upper environment 110 to the data store 160 of the lower environment 145 in real-time, or in near real-time. The amount of delay between a message being written to the data store 125 at the upper environment 110 and the message being de-identified by the de-identifier 120 and written to the data store 160 of the lower environment 145 can be low or negligible. For example, the message can be streamed across environments 110 or 145 in 1 second to 10 seconds, 500 milliseconds to 1 second, 100 milliseconds to 500 milliseconds, 10 milliseconds to 100 milliseconds, 1 millisecond to 10 millisecond.

The upper environment 110 can include at least one record module 130. The record module 130 can be activated or deactivated with a flag. The record module 130 can turn on recording of a live production data stream as the de-identifier 120 de-identifies the data in real-time. The record module 130 can record messages of a feed or facility to allow for debugging of messages that may fail. The record module 130 can begin recording messages responsive to the flag being enabled. The record module 130 can stop recording the messages responsive to the flag being disabled. The record module 130 can control the recording of specific sets of messages at different levels of granularity (e.g., stream level, feed level, or source level). For example, the record module 130 can record messages of a particular stream of one or multiple streams. A message steam can be a real-time flow of messages aggregated through an event store, such as a topic, which allows the de-identifier 120 to become a consumer or recorder of the message stream. A message feed within the message stream can be a collection of sources within a message feed. Some examples are a test generator feed, a range of Internet protocol (IP) address, a named feed such as a data aggregator which is forwarding on data from lots of sources. A message source or facility within a given feed, e.g., an HL7 MSH.4 sending facility, a transmission control protocol (TCP) port, or an IP address which uniquely identities a source of data.

At least one feature flag or other indicator can toggle recording by the record module 130 and de-identification of the de-identifier 120. Responsive to the feature flag being toggled on, the de-identifier 120 can start processing a stream of messages written to the data store 125. In some examples, the de-identifier 120 does not de-identify the messages and the record module 130 does not record the messages if the flag is not activated. The system 100 may not record or de-identify messages unless there is an active de-identification configuration that matches the data within the stream. Furthermore at least one flag, indicator, or configuration can specify which feeds within a message stream the de-identifier 120 should de-identify, and which feeds within a message stream should not be de-identified. Furthermore at least one flag, indicator, or configuration can specify which sources should be de-identified, and which sources should not be de-identified. For example, data could indicate that only a specific source should be de-identified. Responsive to receiving or reading this data, the de-identifier 120 can only de-identify messages from that source and the record module 130 can record only messages of that source, and all other messages in the stream or feed can be ignored.

Because the de-identifier can hash all values of messages of the data store 125, except components excluded from hashing, in combination with the applying default formatters allows for the system 100 to properly de-identify messages. Because metadata of the messages may be excluded from the hashing, the de-identified message will continue to meet specific criteria and be processable by the lower environment 145.

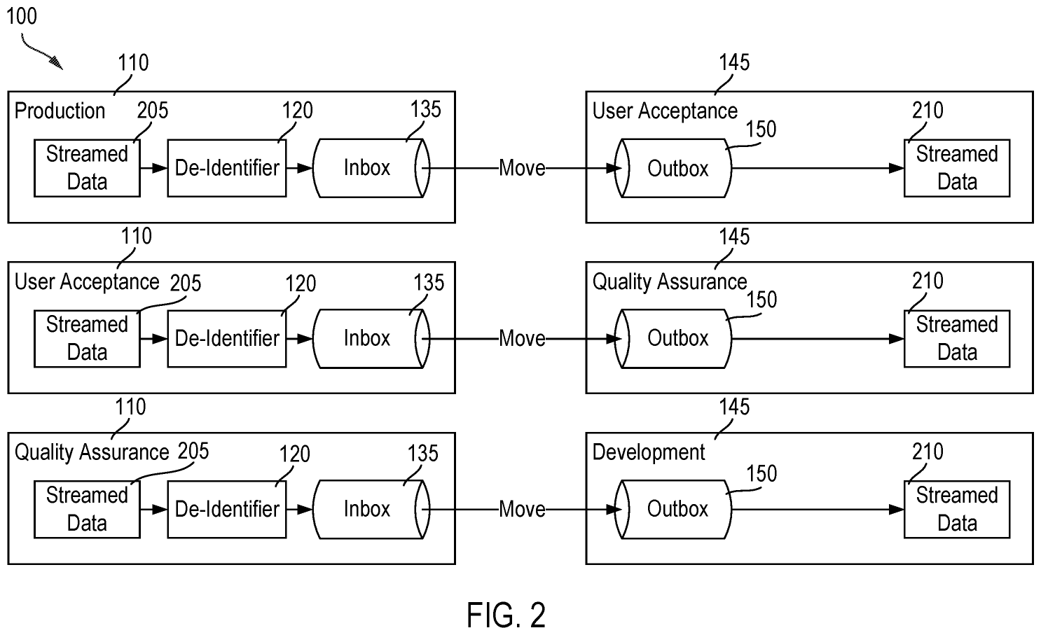

Referring now to FIG. 2, among others, a system 100 including de-identification of a medical message stream communicated between environments is shown. The system 100 is shown to receive streamed data 205. For example, each of the production environment 110, the user acceptance environment 110, and the quality assurance environment 110 can receive streamed data 205. The streamed data 205 can be the data written to the data store 125. Each of the user acceptance environment 145, the quality assurance environment 145, and the development environment 145 can output the streamed data 210, which can be data written to the data store 160.

The system 100 can stream data from an upper environment 110 to a lower environment 145 via an inbox-outbox pattern and seamlessly down through a chain of environments, e.g., from a production environment 110 to a user acceptance environment 145, from a user acceptance environment 145 to a quality assurance environment 145, from a quality assurance environment 110 to a development environment 145. Messages can be chained down through lower environments in real-time by enabling a feature flag in the lower environment and enabling the corresponding de-identification configuration. Messages that are de-identified in a higher environment can be tagged with a meta-data property or value to indicate that the message has already been de-identified. This can ensure that the upper environments 110 and the lower environments 145 do not de-identify the message multiple times, and that the message is only de-identified once. The system 100 can allow for the de-identification of large volumes of data over time and in real-time without placing significant delays or processing over head on the environment data flows. The lower environments 145 can accurately mirror the peaks and troughs of the production data, allowing better and more accurate testing in the lower environment. The system 100 can allows lower environments 145 to be seeded with production like data at the ingress points where data flows into the systems, allowing for improved end-to-end testing strategies.

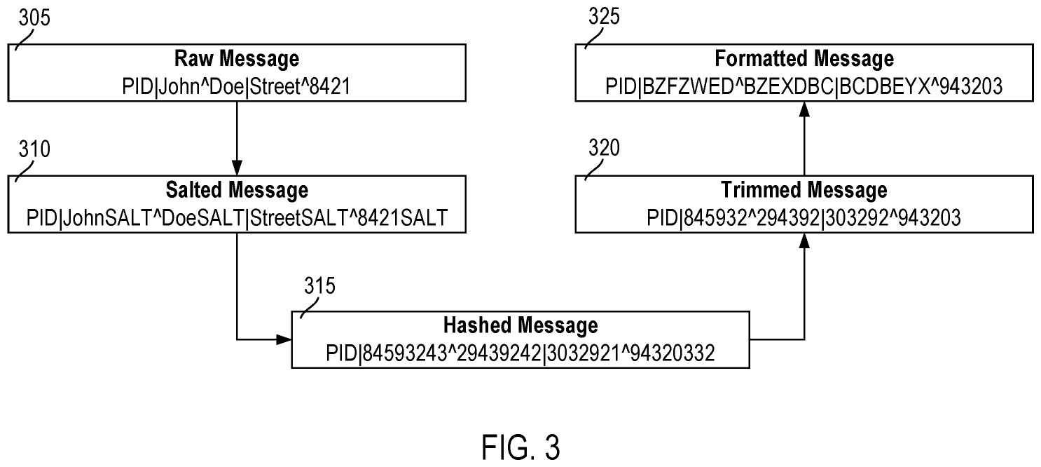

Referring now to FIG. 3, among others, de-identification of a message is shown. FIG. 3 illustrates the transformation and formatting of the streamed messages 305 by the de-identifier 120. Raw message 305 illustrates an example message that the de-identifier 120 can receive from the data store 125. The raw message 305 can include PII, e.g., a first name, a last name, an address, a date, a phone number, a zip code, a street, a data of birth, a Medicare Beneficiary Identifier (MBI), an insurance name, an insurance identifier.

The raw message 305 can be salted to form the salted message 310. The de-identifier 120 can add a salt value to each component of the raw message 305. For example, the de-identifier 120 can parse the raw message 305 into multiple components, e.g., a name component and a street component in FIG. 3. Each component can be concatenated with a salt value. The salt value can be added to the start or the end of the component. The de-identifier 120 can hash the salted message 310 to generate a hashed message 315. The de-identifier 120 can hash each component individually with each respective salt value.

The de-identifier 120 can trim the hashed message 315 to generate the trimmed message 320. The de-identifier 120 can count the number of characters in each component. The de-identifier can compare the number of characters in each component to a threshold. The de-identifier 120 can determine if the number of characters is greater than the threshold, less than the threshold, or equal to the threshold. If the number of characters is equal to the threshold, the de-identifier may not modify the component. If the number of characters is greater than the threshold, the de-identifier 120 can remove or discard excess characters of the component, e.g., remove one or more ending characters or beginning characters such that the number of characters in the component equals the threshold. If the number of characters is less than the threshold, the de-identifier 120 can add additional characters, e.g., add one or more ending characters or beginning characters to the component such that the number of characters in the component equals the threshold.

The de-identifier 120 can format the trimmed message 320 to generate the formatted message 325. The de-identifier 120 can identify a type of each component of the raw message 305 or a type of the PII of each component. For example, the de-identifier 120 can identify that a component represents a first name, a last name, an address, a date, a phone number, a zip code, a street, a data of birth, a Medicare Beneficiary Identifier (MBI), an insurance name, an insurance identifier. The de-identifier 120 can select a formatting rule from multiple different formatting rules. Each formatting rule can be specific for formatting components of at least one component or PII type. For example, a formatting rule may be specific to formatting phone numbers. Another formatting rule may be specific to formatting email addresses. The formatting rules can include formatting rules for residential addresses, email addresses, Booleans, phone numbers, uniform resource locators (URLs), dates, floating point numbers, globally unique identifiers (GUIDs), medicare beneficiary identifiers (BMIs), number rules, string rules, etc. Various formatting rules can be added or implemented for various use cases.

For each component, the de-identifier 120 can select a formatting rule and format the component with the selected formatting rule. Formatting the hashed or transformed components can improve the meaning and readability of the overall de-identified message. For example, the hashed messages can be enriched with metadata that allows a system or reader to quickly identify what type of PII the hashed component represents. Formatting the rule can include adding characters to the component, removing characters from the component, or replacing characters of the component with other characters. The de-identifier 120 can format the component after the component has been transformed, e.g., hashed.

For example, an address rule can replace hashed characters of a street with letters and the word “Street.” For example, a resulting transformed component formatted with the address rule could be “XKSME Street.” For example, a date rule can replace hashed characters of a date with numbers and organize the numbers in a year, month, date format. For example, a resulting transformed component formatted with the date rule could be “20200816.” For example, a phone number rule can replace hashed characters of a phone number with numbers. Furthermore, the phone number rule can add parenthesis before and after the first three numbers of the component. Furthermore, the phone number rule can add a space or dash between the sixth and seventh character. For example, a resulting transformed component formatted with the phone number rule could be “(555)203 0321.” For example, a BMI rule could format a component with numbers and letters A-Z, e.g., a resulting component formatted with the BMI rule could be “1DH3TG2RQ35.”

The de-identifier 120 can detect that there is no formatting rule for a personal identifying information type for the components of the trimmed message 320. If the de-identifier 120 is unable to identify the type of PII that a component represents, the de-identifier 120 can apply formatting rules based on the type of data type of the component, e.g., floating-point, number, string, etc. For example, the de-identifier 120 can detect a data type of the component. For example, the de-identifier 120 can read a data type of the original component of the raw message 305 and select a default formatting rule. The default formatting rule can be a formatting rule specific to the underlying data type of the component. The de-identifier 120 can select from multiple different default formatting rules, each default formatting rule for one data type. For example, if the component of the raw message 305 is a floating-point, the de-identifier can apply a floating-point rule. The de-identifier 120 can add a period into the transformed component, e.g., the resulting formatted component can be “30.43.” If the component of the raw message 305 is a number, the de-identifier can apply a number rule. The number rule can include replacing characters of the hashed component with number characters. If the component is a string, the de-identifier 120 can apply a string rule. For example, the string rule can format the message with letters A-Z, e.g., an example formatted component with the string rule could be “DSKGNSA.”

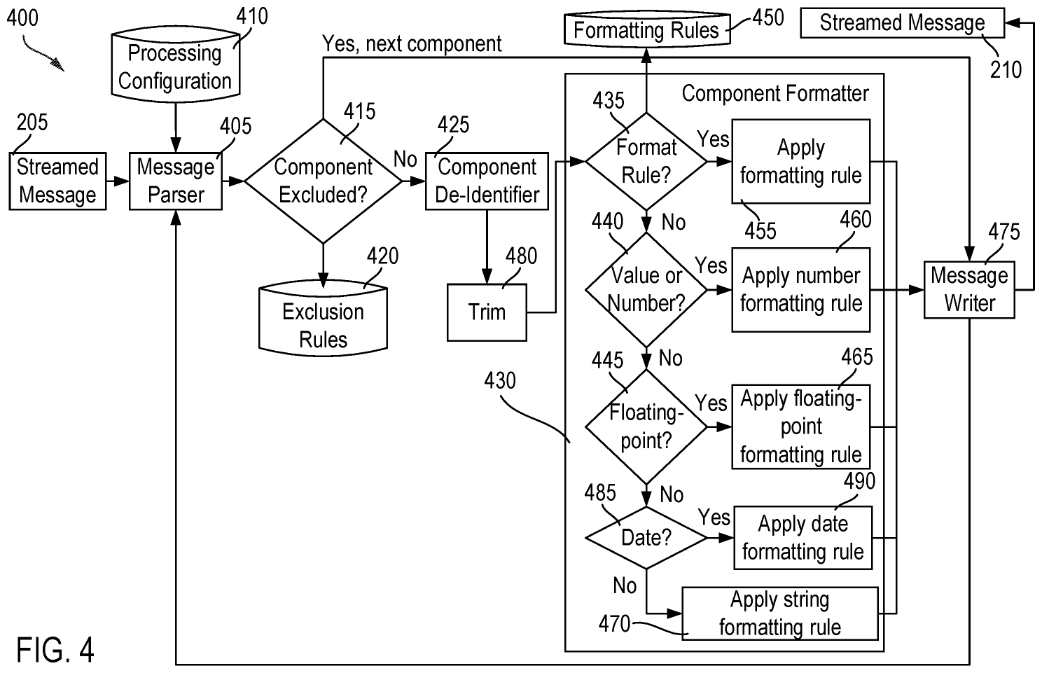

Referring now to FIG. 4, among others, an example method 400 of de-identification of a medical message stream is shown. FIG. 4 includes ACTS of a method 400, but also includes representations of messages and the components of the de-identifier 120. For example, the components of the de-identifier 120 in FIG. 4 (e.g., a message parser 405, a component de-identifier 425, a component formatter 430, and a message writer 475) can perform the ACTS of the method 400. The de-identifier 120 can process large volumes of data in real-time, and each layer of the de-identifier can automatically scale horizontally and add additional processing workloads to manage increases to the volume of data to be de-identified. The message parser 405 and the message writer 475 can be implemented for one or multiple message streams that pass messages according to a protocol or standard. The de-identifier 120 (or component of the de-identifier 120) can be implemented for each message stream. For example, a first instantiation of the de-identifier 120 can be run for a first message stream, while a second instantiation of the de-identifier 120 can be run for a second message stream.

The method 400 can include receiving streamed messages 205 at a message parser 405. The message parser 405 can be specific to a particular message format of the streamed messages 205, for example, the message parser 405 can be a JSON parser to parse JSON message formats, an HL7 parser to parse HL7 messages, an XML parser to parse XML message formats, a FHIR parser to parse FHIR messages, or Binary message parser to parse Binary messages.

The message parser 405 can parse received messages into a model. The message parser 405 can implement parsing based on rules, configurations, or settings of a processing configuration 410. The processing configuration 410 can identify frames, locations, addresses, or positions within the streamed message 205 where components or PII data exist, start, or end. The processing configuration 410 can be a static file, or database. The processing configuration 410 can be a dynamic file, e.g., the processing configuration 410 can be updated over time to allow for new message formats to be parsed by the message parser 405. The message parser 405 can, for a received streamed message 205, identify a format of the message 205. Based on the identified format, the message parser 405 can retrieve or read a configuration for parsing the message 205 from the processing configuration 410. Responsive to retrieving the configuration, the message parser 405 can parse the message into components based on the retrieved configuration.

The message parser 405 can parse the streamed message 205 into multiple components. At ACT 415, the de-identifier 120 can determine, for each component, whether to exclude the component from de-identification, or to de-identify the component. For example, the de-identifier 120 can determine whether each component should be written in the clear or not. The de-identifier 120 can default to transforming or hashing every component of the message, unless an exclusion rule 420 indicates that a particular component should be excluded from transformation. In this regard, no PII information is shown in the clear in the streamed message 210, unless explicitly indicated.

The exclusion rules 420 can indicate, for a particular message format, that a component should be excluded from de-identification. The de-identifier 120 can select one or more exclusion rules specific to a format of the streamed message 205 and process the streamed message 205 with the selected exclusion rule 420 to process each component of the streamed message 205 and determine whether the component should be de-identified or excluded from de-identification. The exclusion rules 420 can each be linked to a particular type of message format, e.g., one or a set of exclusion rules 420 can be stored for a first message format, one or a set of exclusion rules 420 can be stored for a second message format, etc. The de-identifier 120 can process each component individually, or process all of the components together, with the selected exclusion rules 420 to determine if each component should be excluded from de-identification 120.

Some information of the streamed message 205 should not be de-identified. For example, some information may need to be excluded from de-identification to preserve the meaning of the message, where the information is not sensitive or personally identifiable in nature. For example, metadata of the message, which may be necessary for understanding the message in general, may be excluded from transformation. An excluded message component could be the HL7 V2 MSH segment. The MSH segment can define the intent, source and syntax of the HL7 message.

The de-identifier 120 can provide an excluded component to the message writer 475. The excluded component can be passed to the message writer 475 without change. The message writer 475 can write the message to the streamed message 210 responsive to receiving the excluded component. The message writer 465 can write the excluded component in the clear, e.g., without any transformation or hashing. The excluded component can be written in its original form, e.g., no value or character of the excluded component may be modified, changed, removed, or replaced and no new value or character added to the excluded component, responsive to identifying that a message component is excluded from de-identification. Because the de-identifier 120 de-identifies all message components, even if new types or formats of messages of varying sizes are added to the message stream, the de-identifier 120 still de-identifies all PII of the new message because the de-identifier 120 de-identifies all message components unless the exclusion rules 420 explicitly indicate otherwise.

If a component is not excluded at ACT 415, the component can be de-identified by the component de-identifier 425. The component de-identifier 425 can de-identify each component of the streamed message 205 as the components are parsed from the streamed message 205 by the message parser 405. The component de-identifier 425 can hash each component with a hashing algorithm. The component de-identifier 425 can hash each component individually as the components are ready for hashing, e.g., responsive to the component being parsed from the streamed message 205. The component de-identifier 425 can apply a SHA512 algorithm, a SHA-256 algorithm, a MD5 algorithm, a RIPEMD-160 algorithm, a Whirlpool algorithm, or any other type of hashing algorithm. The de-identifier 425 can apply a non-reversible securing hashing function to the components. At ACT 480, the components can be trimmed. For example, characters or values can be added or removed from the components such that the components meet a predefined character length.

The de-identified and trimmed components can be provided to a component formatter 430. Data formatting can be applied by the component formatter 430 to the hashed values of the components to ensure the message continues to adhere to a message standard or protocol rule. The component formatter 430 can format the de-identified component according to formatting rules 450. The component formatter 430 can select a formatting rule or a set of formatting rules based on an original type of the data present in the original component of the streamed message 205 (e.g., the component of the streamed message 205 before it was de-identified). At ACT 435, the component formatter 430 can select a formatting rule 450. The component formatter 430 can search the formatting rules 450 to determine whether a formatting rule 450 exists for a type of the component. Each formatting rule 450 can be linked to a different component type. Responsive to identifying a formatting rule 450 linked to a particular type for the component, the component formatter 430 can apply the formatting rule 450 at ACT 455.

If the component formatter 430 does not identify a formatting rule from the formatting rules 450, the component formatter 430 can proceed to ACT 440. At ACT 440, the component formatter 430 can determine if the original component of the streamed message 205 (e.g., the component of the streamed message 205 before it was de-identified) is a number type. If the original component is a number type, then the component formatter 430 can apply a number formatting rule at ACT 460. At ACT 445, the component formatter 430 can determine if the original component of the streamed message 205 is a floating-point type. If the original component is a floating-point type, then the component formatter 430 can apply a floating-point formatting rule at ACT 465. The floating-point formatting can maintain the same level of accuracy after the decimal point. At ACT 470, the component formatter 430 can determine if the original component of the streamed message 205 is a string type. If the original component is a string type, then the component formatter 430 can apply a string type formatting rule at ACT 470. At ACT 485, the component formatter 430 can determine if the original component of the streamed message 205 is a date type. If the original component is a date type, then the component formatter 430 can apply a date type formatting rule at ACT 490. ACT 470 can be a default step, e.g., if there is no formatting rule at ACT 435, no value or number at ACT 440, no floating-point at ACT 445, or no date at ACT 485, the ACT 470 can be performed. The number formatting rule, the floating-point formatting rule, and the string formatting rule can be default rules that are applied if the component formatter 430 cannot find a formatting rule 450 (or the formatting rule 450 is not stored) for the particular type of the original component of the streamed message 205.

The message writer 475 can write the streamed message 210 based on the components formatted by the component formatter 430 and/or the components excluded from transformation. As soon as a component is available to the message writer 475 to be written to the streamed message 210, the message writer 475 can write the component to the streamed message 210. In this regard, the streamed message 210 can be written, generated, or created as the de-identifier 120 parses the streamed message 205, checks for excluded components, de-identifies the components with the component de-identifier 425, and formats the components with the component formatter 430.

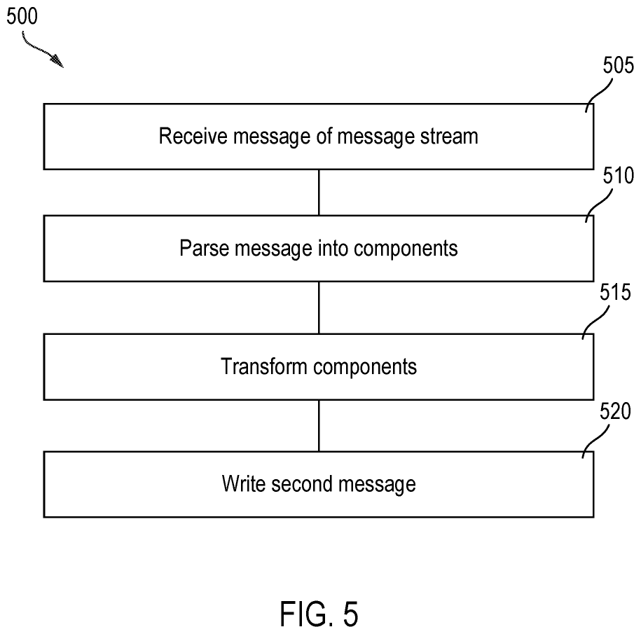

Referring now to FIG. 5, among others, a method 500 of de-identification of a medical message stream is shown. At least a portion of the method 500 can be performed by the upper environment 110, the lower environment 145, the de-identifier 120, the data store 125, the record module 130, the inbox 135, the workflow service 140, the outbox 150, the connector 155, the data store 160, or the computing system 105. The method 500 can include an ACT 505 of receiving a message of a message stream. The method 500 can include an ACT 510 of parsing a message into components. The method 500 can include an ACT 515 of transforming components. The method 500 can include an ACT 520 of writing a second message.

At ACT 505, the method 500 can include receiving, by the computing system 105, a message of a message or event stream. For example, the upper environment 110 can receive a message of a message stream via a data store 125. The de-identifier 120 can receive the message of the message stream via the data store 125. For example, the de-identifier 120 can be registered or subscribed to the data store 125, and can receive or read the messages of the message stream via the data store 125.

At ACT 510, the method 500 can include parsing, by the computing system 105, a message into components. For example, the de-identifier 120 can parse the message received at ACT 505 into one or multiple parts, pieces, segments, or components. Each component can be, represent, or include PII data. The message parser 405 can parse the message according to a processing configuration 410 specific to a type of the message.

At ACT 515, the method 500 can include transforming, by the computing system 105, components. For example, the component de-identifier 425 can transform each component to de-identify the components. For example, the de-identifier 425 can hash each component according to a hashing algorithm. Furthermore, the de-identifier 120 can determine whether to exclude one or more of the components from de-identification. For example, information that describes the purpose, source, or intent of the message can be excluded from de-identification. The de-identifier 425 can provide the excluded components to the message writer 475 to write the excluded components to an output message in the clear.

Furthermore, the method 500 can include formatting the transformed components. For example, the transformed components can be formatted by the component formatter 430 according to formatting rules 450. Each formatting rule 450 can be specific to a type of the original component or what information the original component represented. If no formatting rule 450 is available or configured to format a component of a specific type, the component formatter 430 can apply one or more default formatting rules. The default formatting rules can format the component based on the underlying data type of the original component, e.g., number, floating-point, or string.

At ACT 520, the method 500 can include writing, by the computing system 105, a second message. For example, the message writer 475 can write transformed components to a new message. The message writer 475 can write the message asynchronously. For example, the message writer 475 can write transformed or excluded components to the new message as soon as the components are available, instead of waiting for all components to be available and writing all of the components of the new message at once. For example, responsive to a component being transformed and formatted, the message writer 475 can write the transformed component to the new message. For example, responsive to a component being identified as excluded from transformation, the message writer 475 can write the excluded component to the new message. In this regard, the message writer 475 can write the components as the components are available from left to right, or right to left, if the message is a series of frames or segments. If the message writer 475 writes a column, the message writer 475 can write the components as the components are available for writing from top to bottom, or from bottom to top.

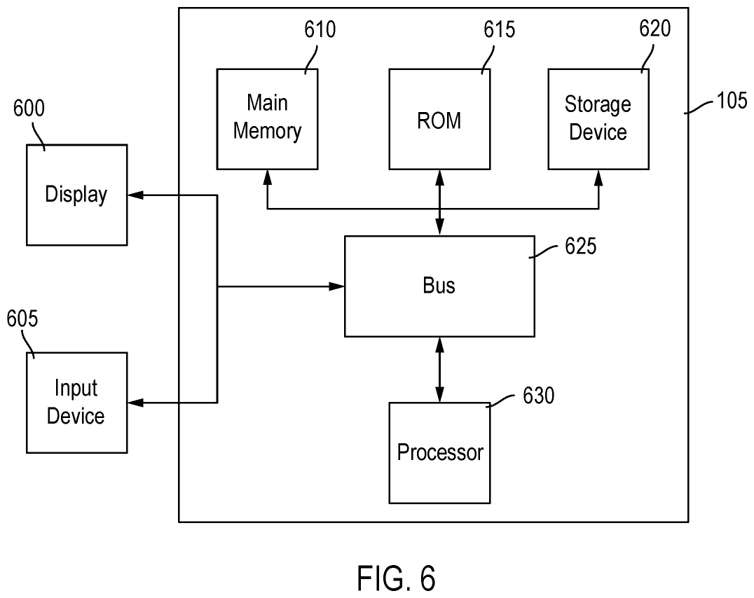

Referring now to FIG. 6, among others, an example block diagram of the computing system 105 is shown. The computing system 105 can include or be used to implement a data processing system or its components. The architecture described in FIG. 6 can be used to implement the computing system 105, or any other computing device. The computing system 105 can include at least one bus 625 or other communication component for communicating information and at least one processor 630 or processing circuit coupled to the bus 625 for processing information. The computing system 105 can include one or more processors 630 or processing circuits coupled to the bus 625 for processing information. The computing system 105 can include at least one main memory 610, such as a random access memory (RAM) or other dynamic storage device, coupled to the bus 625 for storing information, and instructions to be executed by the processor 630. The main memory 610 can be used for storing information during execution of instructions by the processor 630. The computing system 105 can further include at least one read only memory (ROM) 615 or other static storage device coupled to the bus 625 for storing static information and instructions for the processor 630. A storage device 620, such as a solid state device, magnetic disk or optical disk, can be coupled to the bus 625 to persistently store information and instructions.

The computing system 105 can be coupled via the bus 625 to a display 600, such as a liquid crystal display, or active matrix display. The display 600 can display information to a user. An input device 605, such as a keyboard or voice interface can be coupled to the bus 625 for communicating information and commands to the processor 630. The input device 605 can include a touch screen of the display 600. The input device 605 can include a cursor control, such as a mouse, a trackball, or cursor direction keys, for communicating direction information and command selections to the processor 630 and for controlling cursor movement on the display 600.

The processes, systems and methods described herein can be implemented by the computing system 105 in response to the processor 630 executing an arrangement of instructions contained in main memory 610. Such instructions can be read into main memory 610 from another computer-readable medium, such as the storage device 620. Execution of the arrangement of instructions contained in main memory 610 causes the data computing system 105 to perform the illustrative processes described herein. One or more processors in a multi-processing arrangement can be employed to execute the instructions contained in main memory 610. Hard-wired circuitry can be used in place of or in combination with software instructions together with the systems and methods described herein. Systems and methods described herein are not limited to any specific combination of hardware circuitry and software.

Although an example computing system has been described in FIG. 6, the subject matter including the operations described in this specification can be implemented in other types of digital electronic circuitry, or in computer software, firmware, or hardware, including the structures disclosed in this specification and their structural equivalents, or in combinations of one or more of them.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiation in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C#, PROLOG, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices include cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or example, and references to “an implementation,” “some implementations,” “one implementation” or the like are not necessarily mutually exclusive and are intended to indicate that a particular feature, structure, or characteristic described in connection with the implementation may be included in at least one implementation or example. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

What is claimed is:

1. A system comprising:
one or more memory devices storing instructions thereon, that, when executed by one or more processors, cause the one or more processors to:
receive a message of a message stream, the message comprising a medical data;
parse the message into a plurality of components representing personal identifying information of the medical data;
transform the plurality of components to de-identify the personal identifying information; and
write a second message based on the plurality of transformed components.

2. The system of claim 1, wherein the instructions cause the one or more processors to:
stream the second message, and a plurality of second messages comprising transformed components, to a second system.

3. The system of claim 1, wherein the instructions cause the one or more processors to:
transform a first component of the plurality of components;
write the transformed first component to the second message;
transform a second component of the plurality of components as the transformed first component is written to the second message; and
write the transformed second component to the second message.

4. The system of claim 1, wherein the instructions cause the one or more processors to:
write the second message based on the plurality of transformed components as the plurality of components are transformed.

5. The system of claim 1, wherein the instructions cause the one or more processors to:
identify a component of the plurality of components that is excluded from transformation; and
write the component to the message in the clear.

6. The system of claim 1, wherein the instructions cause the one or more processors to:
add salt values to the plurality of components; and
transform the plurality of components with the salt values.

7. The system of claim 1, wherein the instructions cause the one or more processors to:
hash a component of the plurality of components;
determine that a number of characters of the hashed component is greater than a threshold; and
discard at least one character of the characters of the hashed component to cause the number of characters of the hashed component to equal the threshold.

8. The system of claim 1, wherein the instructions cause the one or more processors to:
identify that a component of the plurality of components represents a type of personal identifying information of a plurality of types of personal identifying information;
select a rule for the type from a plurality of rules, the plurality of rules linked with the plurality of types of personal identifying information; and
format a transformed component of the plurality of transformed components based on the rule by adding at least one additional character to the transformed component or replacing at least one character of the transformed component with a predefined type of character.

9. The system of claim 1, wherein the instructions cause the one or more processors to:
add the second message to an inbox of a first environment, the inbox comprising a plurality of second messages;
receive a trigger; and
move the plurality of second messages from the inbox to an outbox of a second environment.

10. The system of claim 1, wherein the instructions cause the one or more processors to:
detect that there is no formatting rule for a personal identifying information type a component of the plurality of components;
detect a data type of the component; and
format a transformed component of the plurality of transformed components based on a default rule of a plurality of default rules, the plurality of default rules linked to a plurality of data types.

11. A method, comprising:
receiving, by one or more processing circuits, a message of a message stream, the message comprising a medical data;
parsing, by the one or more processing circuits, the message into a plurality of components representing personal identifying information of the medical data;
transforming, by the one or more processing circuits, the plurality of components to de-identify the personal identifying information; and
writing, by the one or more processing circuits, a second message based on the plurality of transformed components.

12. The method of claim 11, comprising:
transforming, by the one or more processing circuits, a first component of the plurality of components;
writing, by the one or more processing circuits, the transformed first component to the second message;
transforming, by the one or more processing circuits, a second component of the plurality of components as the transformed first component is written to the second message; and
writing, by the one or more processing circuits, the transformed second component to the second message.

13. The method of claim 11, comprising:
identifying, by the one or more processing circuits, a component of the plurality of components that is excluded from transformation; and
writing, by the one or more processing circuits, the component to the message in the clear.

14. The method of claim 11, comprising:
adding, by the one or more processing circuits, salt values to the plurality of components; and
transforming, by the one or more processing circuits, the plurality of components with the salt values.

15. The method of claim 11, comprising:
hashing, by the one or more processing circuits, a component of the plurality of components;
determining, by the one or more processing circuits, that a number of characters of the hashed component is greater than a threshold; and
discarding, by the one or more processing circuits, at least one character of the characters of the hashed component to cause the number of characters of the hashed component to equal the threshold.

16. The method of claim 11, comprising:
identifying, by the one or more processing circuits, that a component of the plurality of components represents a type of personal identifying information of a plurality of types of personal identifying information;
selecting, by the one or more processing circuits, a rule for the type from a plurality of rules, the plurality of rules linked with the plurality of types of personal identifying information; and
formatting, by the one or more processing circuits, a transformed component of the plurality of transformed components based on the rule by adding at least one additional character to the transformed component or replacing at least one character of the transformed component with a predefined type of character.

17. The method of claim 11, comprising:
adding, by the one or more processing circuits, the second message to an inbox of a first environment, the inbox comprising a plurality of second messages;
receiving, by the one or more processing circuits, a trigger; and
moving, by the one or more processing circuits, the plurality of second messages from the inbox to an outbox of a second environment.

18. The method of claim 11, comprising:
detecting, by the one or more processing circuits, that there is no formatting rule for a personal identifying information type of a component of the plurality of components;
detecting, by the one or more processing circuits, a data type of the component; and
formatting, by the one or more processing circuits, a transformed component of the plurality of transformed components based on a default rule of a plurality of default rules, the plurality of default rules linked to a plurality of data types.

19. One or more storage medium storing instructions thereon, that, when executed by one or more processors, cause the one or more processors to:
receive a message of a message stream, the message comprising a medical data;
parse the message into a plurality of components representing personal identifying information of the medical data;
transform the plurality of components to de-identify the personal identifying information; and
write a second message based on the plurality of transformed components.

20. The one or more storage medium of claim 19, wherein the instructions cause the one or more processors to:
transform a first component of the plurality of components;
write the transformed first component to the second message;
transform a second component of the plurality of components as the transformed first component is written to the second message; and
write the transformed second component to the second message.
